(19) United States
(12) Patent Application Publication
(10) Pub. No.: US 2019/0228439 A1
(43) Pub. Date: Jul. 25, 2019

(54) DYNAMIC CONTENT GENERATION BASED ON RESPONSE DATA

(71) Applicant: Vungle, Inc., San Francisco, CA (US)

(72) Inventors: Ryan John Leslie Anthony, Los Angeles, CA (US); Simon John Crowhurst, London (GB); Martin Jeffrey Price, San Francisco, CA (US)

(21) Appl. No.: 16/249,579

(22) Filed: Jan. 16, 2019

Related U.S. Application Data
(60) Provisional application No. 62/619,699, filed on Jan. 19, 2018.

(51) Int. Cl. G06Q 30/02; G16H 30/20

(52) U.S. Cl. CPC G06Q 30/0271; G16H 30/20

(57) ABSTRACT

Methods and systems are described for collecting response data, such as electroencephalography data, functional magnetic resonance imaging data, galvanic skin response data, heart rate data, body temperature data, eye tracking data, face tracking data, head tracking data, etc., as users receive a presentation of digital content and then utilizing that response data to dynamically produce or revise other digital content, such as advertisements, to elicit a specific user response and an expected engagement with the digital content, such as the advertisement.

### PRIORITY CLAIM

This application claims priority to U.S. Provisional Patent Application No. 62/619,699, filed Jan. 19, 2018, and titled “Dynamic Content Generation Based on Response Data,” the contents of which are incorporated by reference herein in their entirety.

### BACKGROUND

With the continued increase in mobile device usage and the availability to digital content, advertising is shifting from generic print advertising to user specific and targeted digital advertising. However, this shift has resulted in advertisers having more difficulty developing targeted advertisements for the wide variety of consumers and their preferences. Likewise, consumers have become more inundated with advertisements making it even more difficult for advertisements to stand out and be engaging to the consumers.

### BRIEF DESCRIPTION OF THE DRAWINGS

FIG. 1 is a block diagram of a system for communicating with user devices, publishers, and advertisers, in accordance with described implementations.

FIG. 2 is a diagram illustrating a plurality of controlled groups utilized to collect response data in a controlled environment, in accordance with described implementations.

FIG. 3 is a more detailed view of a controlled group participant viewing digital content and response data being collected, in accordance with described implementations.

FIG. 4 illustrates user collected metrics produced in response to a user of a controlled group viewing an advertisement, in accordance with described implementations.

FIGS. 5A and 5B illustrate user collected metrics produced in response to a user of a controlled group viewing an advertisement, in accordance with described implementations.

FIGS. 5C and 5D are graphical illustration of an advertisement, in accordance with described implementations.

FIG. 6 is a block diagram of a machine learning system that receives response data, according to described implementations.

FIG. 7 is a block diagram of the machine learning system of FIG. 6, producing output models based on the training inputs, in accordance with described implementations.

FIG. 8 is a block diagram of an example trained machine learning system that produces recommended digital content configurations based on user inputs and the trained models, in accordance with described implementations.

FIG. 9 is a block diagram illustrating the exchange of information between a machine learning system, an ad server system, group metrics, a user device, publishers, an advertiser, and an attributor, in accordance with described implementations.

FIG. 10 is an example response data collection process, in accordance with described implementations.

FIG. 11 is an example digital content presentation process, in accordance with described implementations.

FIG. 12 is an example continuous update of dynamic digital content process, in accordance with described implementations.

FIG. 13 is a pictorial diagram of an illustrative implementation of a server system that may be used for various implementations.

### DETAILED DESCRIPTION

Methods and systems are described for collecting response data, such as electroencephalography (“EEG”) data, functional magnetic resonance imaging (“fMRI”) data, galvanic skin response (“GSR”) data, heart rate data, body temperature data, eye tracking data, face tracking data, head tracking data, macro-expression tracking data, micro-expression tracking data, etc., as users receive a presentation of digital content (e.g., images, video, audio, tactile) and then utilizing that response data to dynamically produce or revise other digital content to elicit a specific user response and an expected engagement with the digital content.

Digital content, as used herein, refers to any type of digital content that may be presented to a user, including but not limited to audio, video, images, tactile or haptic, etc., or any combination thereof. In some implementations, digital content may be a digital advertisement, a digital document (e.g., whitepaper), a digital video clip, a segment of an application, etc. Likewise, in some implementations, one or many different forms of digital content may be presented to users as sensor data is collected in response to that presentation. The collected response data may then be used to produce or revise other forms or types of digital content. For example, in some implementations, users in a controlled environment (discussed below) may be presented with a first form of digital content, such as a series of images or colors, and the response data collected from sensors as the first form of digital content is presented may be utilized to generate or revise a second form of digital content, such as digital advertisements.

In some implementations, one or more controlled environments may be utilized to collect response data from a group of users viewing digital content in the controlled environment, referred to herein generally as a controlled group of users or a controlled group. In such an environment, various aspects, such as the environmental conditions (lighting, temperature, noise, etc.), may be controlled as users receive presentations of digital content. Likewise, one or more sensors, such as EEG sensors, fMRI sensors, GSR sensors, skin temperature sensors, cameras, accelerometers, gyroscopes, etc., may be used to collect response data for each user as the user receives the presentation.

The collected response data are representative of a user's conscious and/or non-conscious (or sub-conscious) responses to aspects or variables included in the presented digital content. Responses may include, for example, user primal responses (also referred to herein as system 1 responses), which are responses to different colors, light, and sound, and secondary response (also referred to herein as system 2 responses) which are responses produced with respect to reading, writing, and logic.

For example, EEG data from an EEG sensor may be utilized as a measure of cognitive-affective processing in absence of behavioral responses. As one example, peaks in brain activity indicated by the EEG sensor may be indicative of interest in a corresponding portion of digital content that is presented. Eye tracking data produced from video data generated by a camera may be utilized as a measure of visual attention patterns on the overall digital content and specific variables (e.g., branding) within the digital content. GSR data and/or body temperature data from GSR sensors and/or temperature sensors may be utilized as a measure of emotional arousal to different aspects or variables of the digital content. Facial expression data, such as micro-expressions and/or macro-expressions, determined from the processing of video data generated by a camera, may be utilized as a measure of emotional responses (e.g., frustration, joy, fear, anger, trust) to different aspects or variables of the digital content. In still other examples, questionnaire data, derived from a questionnaire completed by the user after receiving a presentation of digital content may provide indicators regarding content retention, user perceived likeability, etc. As will be appreciated, any number and/or type of data may be collected in a controlled environment by one or more sensors and utilized with the disclosed implementations.

Collected response data may be aggregated by a machine learning system and correlated with different variables of the presented digital content, other information known about the user, and behaviors or engagements with the digital content exhibited by the users and/or other users in response to similar variables and/or other digital content. For example, the collected response data, actual user engagement in response to the presented digital content, and user profile information, such as age, demographics, location, etc., may be provided as inputs to the machine learning system. Utilizing the inputs, the machine learning system may be trained to produce models indicating different variables (e.g., text size, color, sounds, objects, etc.) that will produce a desired response by users of a particular user type. Likewise, the desired response may be further correlated with an expected or likelihood of engagement by the user. A user type, as discussed further below, may be any type or group of users having one or more common characteristics. For example, a user type may be defined as users that live in a particular geographic area (e.g., the south), users within a particular age range, users that respond to different variables in a similar manner, etc.

The produced models may be used to refine or revise other existing digital content to improve user responses to digital content and/or to generate new digital content that will produce a desired user response and corresponding likelihood of engagement. Because system 1 and system 2 responses are at a user's core level and mostly uncontrolled or subconscious responses to a stimulus, utilizing models produced based on actual sensor data indicative of those system 1 and/or system 2 responses from users of a particular user type allows digital content to be created that is highly tuned and able to produce the desired system 1 or system 2 response from users of a particular user type. Likewise, because users often behave in a manner consistent with their system 1 and system 2 responses and actual engagement has been modeled in response to presentations of digital content with the different variables (e.g., different advertising variables), likelihood of engagement by a user can be predicted to occur from users of a particular user type based on the digital content created according to a generated model.

The models produced according to the described implementations provide a technical advantage over existing systems by enabling the production of digital content according to those models such that the digital content will produce a predicted and repeatable user response from users of a particular user type, thereby increasing the likelihood of user engagement. For example, models may be used to produce digital content that will elicit particular system 1 or system 2 responses from a user of a particular user type when viewing the digital content. Likewise, user engagement may also be highly correlated with the responses to the presented digital content, thereby increasing the probability that the user will behave (e.g., engage) in a predicted manner.

As discussed further below, any variety of inputs may be utilized with the machine learning system to produce models. For example, any one or more of the following inputs may be considered by a machine learning system in the production of a model for a particular user type: user type information, a user profile of a particular user for which digital content is to be generated and presented, a placement of generated digital content within an application, a user device profile, environmental factors (e.g., time of day, day of week, location of the user device), etc.

As discussed further below, in addition to collecting sensor data in a controlled environment, in some implementations, sensor data may be collected from users in uncontrolled environments as the users are presented with various digital content. For example, digital content, such as an advertisement, may be generated based on a model produced according to the described implementations and presented to a user as the user commutes to work on the bus (an uncontrolled environment). As the user views the digital content, response data may be collected from various sensors associated with the user, such as a camera on the user's phone, heart rate or skin temperature measured from a wearable device worn by the user, etc. The collected response data, environmental conditions present at the time the user views the digital content, and the user's actual engagement in response to the digital content may be collected and utilized as feedback to the machine learning system as in input for that user and/or for users of the user type corresponding to that user. Such feedback may be utilized to adjust or refine models for digital content generated for that user and/or for other users of that user type.

For ease of discussion, the following examples will be described primarily with respect to advertisements as digital content. However, it will be appreciated that other forms of digital content may be utilized with the described implementations as part of the presentation to the user for collection of sensor data and/or as digital content that is revised or created based on produced models. For example, the described implementations may be utilized to collected response data as a first type of digital content is presented to users in a controlled and/or uncontrolled environment, produce models based on that response data, and then utilize those models to generate or revise the same or different forms of digital content.

As used herein, “advertisers” include, but are not limited to, organizations that pay for advertising services including ads on a publisher network of applications and games. “Publishers” provide content for users. Publishers include, but are not limited to, developers of software applications, mobile applications, news content, gaming applications, sports news, etc. In some instances, publishers generate revenue through selling ad space in applications so that advertisers can present advertisements in those applications to users as the users interact with the application.

Advertisement performance can be defined in terms of one or more of click-through rates (CTR), conversion rates, advertisement completion rates, future transactions with the advertiser, post install actions or revenue, etc. In some implementations, advertisement performance may be defined in terms of response data collected from users as the user views the advertisement.

The process in which a user selects an advertisement is referred to as a click-through, which is intended to encompass any user selection of the advertisement. The ratio of a number of click-throughs to a number of times an advertisement is displayed is referred to as the CTR of the ad. A conversion of an advertisement occurs when a user performs a transaction related to a previously viewed advertisement. For example, a conversion may occur when a user views an advertisement and installs, within a defined period of time, an application being promoted in the advertisement. As another example, a conversion may occur when a user is shown an advertisement and the user purchases an advertised item on the advertiser's web site within a defined time period. Except where otherwise noted, click-through, conversion, or other positive engagement by a user with an advertisement is generally referred to herein as an “engagement.”

The ratio of the number of conversions or engagements to the number of times an advertisement is displayed is referred to as the conversion rate. A completion rate is a ratio of a number of video ads that are displayed to completion to a number of video ads initiated on a device. In some examples, advertisers may pay for their advertisements through an advertising system in which the advertisers bid on ad placement on a cost-per-click (CPC), cost-per-mille clicks (CPM), cost-per-completed-view (CPCV), cost-per-action (CPA), and/or cost-per-install (CPI) basis. A mille represents a thousand impressions.

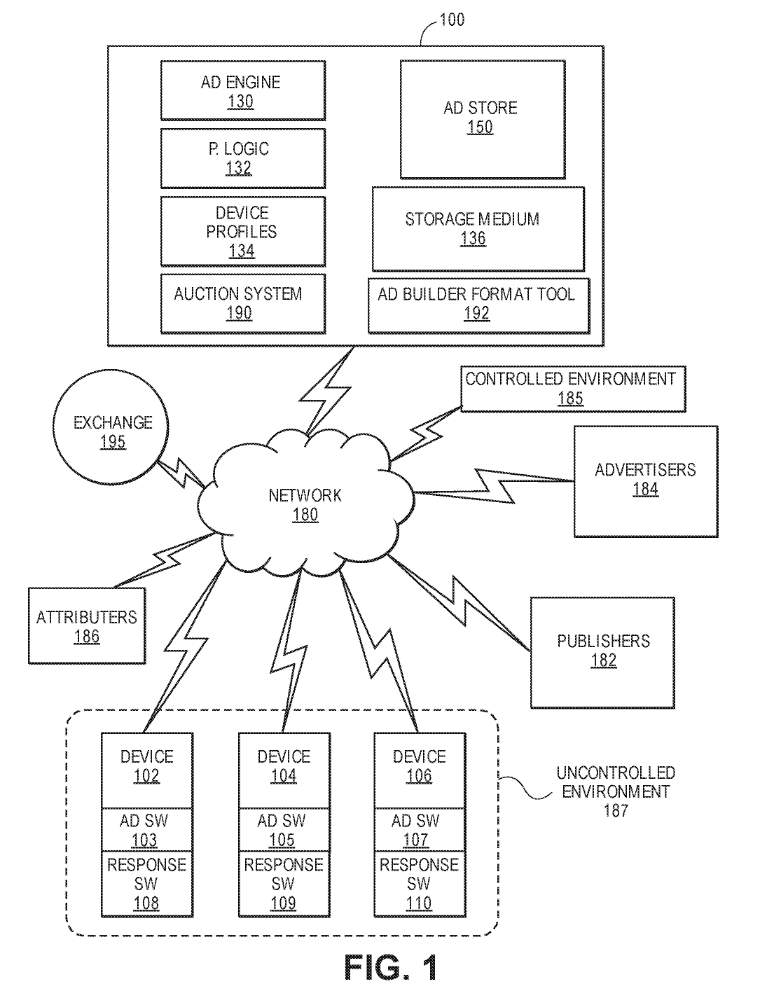

FIG. 1 is a block diagram of an ad server system 100 for communicating with user devices 102, 104, 106, publishers 182, advertisers 184, and one or more controlled environments 185, in accordance with described implementations.

As illustrated in FIG. 1, the ad server system 100 includes an advertising engine 130, processing logic 132, device profiles 134, storage medium 136, an ad store 150, an ad builder format tool 192, and an auction system 190. The auction system 190 may be integrated with the ad server system 100 or separate from the ad server system 100. The ad server system 100 provides advertising services for advertisers 184 to user devices 102, 104, and 106 (e.g., source device, client device, mobile phone, tablet device, laptop, computer, wearable, connected or hybrid television (TV), IPTV, Internet TV, Web TV, smart TV, satellite device, satellite TV, automobile, airplane, etc.) in an uncontrolled environment 187. A user device profile for a device is based on one or more parameters including location (e.g., GPS coordinates, IP address, cellular triangulation, Wi-Fi information, etc.) of the device, a user profile for a user of the device, and/or categories or types of applications installed on the device. Each user device may include respective advertising services software 103, 105, 107 (e.g., a software development kit (SDK)) that includes a set of software development tools for advertising services including in-application advertising services. The user devices may further include response software 108, 109, 110 that collects sensor data from the user device and/or other user devices associated with the user as advertisements are presented to the user. As will be appreciated, users of the devices 102, 104, 106 may be in an uncontrolled environment, which, as used here, is any environment other than a controlled environment 185, discussed herein, in which a user may interact with and/or use the device 102, 104, 106. For example, an uncontrolled environment may include a user's home, a bus, a park, a shopping mall, or any other location of a user.

In comparison, as discussed further below, a controlled environment 185 is a controlled setting in which advertisements are presented to users under known environmental conditions. For example, a controlled environment may be a room in which the noise, temperature, lighting, etc., is controlled along with the time of day, day of week, week of month, and/or month of year in which advertisements are presented to a user and sensor data collected from sensors present in the controlled environment.

The publishers 182 publish content along with selling advertisement space to advertisers. Attributers 186 may install software (e.g., software development kits of publishers) on client devices and track user interactions or engagement with publisher applications and/or advertisements. The attributers 186 may then share this user data with the ad server system 100 and the appropriate publishers 182 and advertisers 184. The ad server system 100, devices 102, 104, 106, advertisers 184, publishers 182, attributers 186, controlled environments 185, and an ad exchange 195 with third party exchange participants communicate via a network 180 (e.g., Internet, wide area network, WiMAX, satellite, etc.). The third party exchange 195 participants can bid in real time or approximately in real time (e.g., 1 hour prior to an ad being played on a device, 15 minutes prior to an ad being played on a device, 1 minute prior to an ad being played on a device, 15 seconds prior to an ad being played on a device, less than 5 seconds prior to an ad being played on a device, less than 1 second prior to an ad being played on a device) to provide advertising services (e.g., an in-application ad that includes a preview (e.g., video trailer) of an application, in-application advertising campaigns for brand and performance advertisers) for the devices.

In one example, an ad format builder tool 192 dynamically generates and provides advertisements for insertion at an ad placement position within an application for presentation to a user via a user device. The ad format builder tool allows a publisher or developer to create a new custom advertisement campaign, associate content items of the advertisement campaign with various tokens, select templates usable for dynamic creation of advertisements, etc. The ad format builder tool may then utilize the information from the advertisement campaign in conjunction with a model generated from the described implementations to dynamically produce an advertisement for a specific user that will elicit a desired response and increase a likelihood of an engagement by the user with the advertisement.

The ad format builder tool 192 provides a technological improvement to advertisers allowing them to provide content items for advertisement campaigns so that advertisements can be dynamically generated according to a model specific to that user and the existing conditions such that the advertisement will produce specific system 1 and system 2 responses from that user, thereby increasing the likelihood of engagement by the user.

In some implementations, the system 100 includes a storage medium 136 to store one or more software programs, content items, etc. Processing logic 132 is configured to execute instructions of at least one software program to receive an advertising request from a user device 102, 104, 106. An ad request may be sent by a device upon the device having an ad play event for an initiated software application and/or upon initiation of an application in which ads may be presented. The processing logic is further configured to send a configuration file and/or an advertisement to the device in response to the advertising request. The configuration file may include different options for obtaining at least one advertisement (“ad”) to play on the user device during an ad play event. Alternatively, or in addition thereto, the configuration file may identify an advertisement that is stored in a memory (e.g., cache) of the user device, identify an advertisement to be obtained from the ad store 150 of the ad server system 100, identify or provide a dynamically generated ad that is to be presented, and/or indicate that an advertisement is to be obtained from the exchange 195.

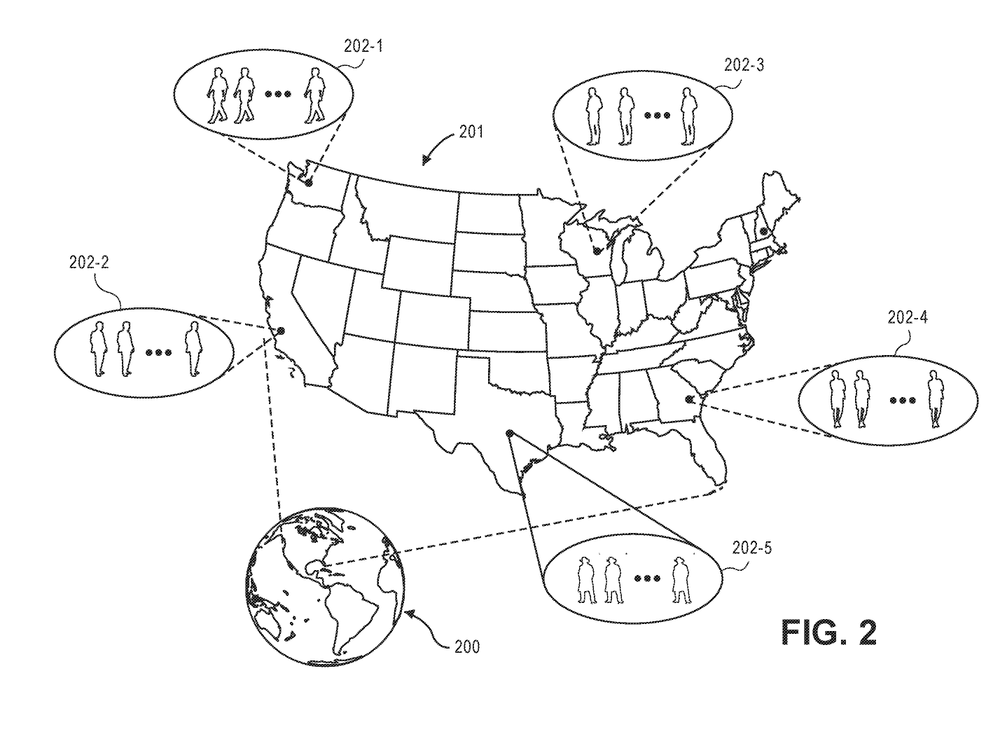

FIG. 2 is a diagram illustrating a plurality of controlled groups 202 utilized to collect response data in a controlled environment, in accordance with described implementations. Controlled groups 202 may be established anywhere in the world 200 and/or in anywhere in the universe that may be populated by humans. The illustrated example, shows the United States of America 201 and five controlled groups 202-1, 202-2, 202-3, 202-4, 202-5. A controlled group may be established at any location and any number of controlled groups may exist. Likewise, controlled groups may be periodically established at the same or different locations to collect response data in response to advertisements and that response data, as used herein, may be utilized to update and revise the machine learning system, the produced models, and the ultimate advertisements.

A controlled group may include any number of users and sensor data may be collected for those users for any length of time. In some implementations, users of a controlled group may all be selected as users of a particular user type. For example, the controlled group 202-5 may be selected to be men living in Texas between the age of 30-45 that are married and earn income between $40,000 USD and $83,000 USD. As will be appreciated, any number and/or type of characteristics may be used to determine a user type for a group of users.

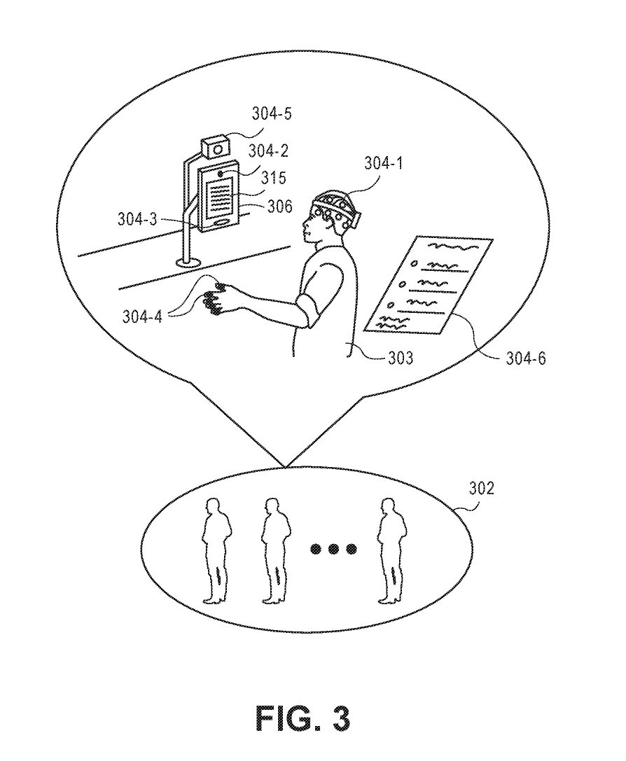

FIG. 3 is a more detailed view of a controlled group participant 303 or user of a controlled group 302 within a controlled environment viewing an advertisement 315 and response data being collected by various sensors 304 within a controlled environment, in accordance with described implementations. In this example, the user 303 is viewing an advertisement 315 presented on a display of a user device 306 that is within the controlled environment. As the user views the advertisement, sensor data is collected from various sensors. Sensors may include, but are not limited to, an EEG sensor 304-1, device camera 304-2, microphone 304-3, GSR sensor 304-4, vision, gaze, face and/or head tracking camera 304-5, fMRI sensor, etc. In other implementations, other sensors may be used in conjunction with or as an alternative to the exemplified sensors 304. Likewise, in some implementations, data may be collected from a questionnaire 304-6 or other input/form that is completed by the user 303 after the user has viewed the advertisement.

As the user 303 views the advertisement and sensor data are collected from the various sensors 304, the sensor data is correlated in time with the presentation of the advertisement. Based on the time correlation between the different sensor data and the advertisement, user responses may be determined and the advertisement variable present at the time of the determined responses identified.

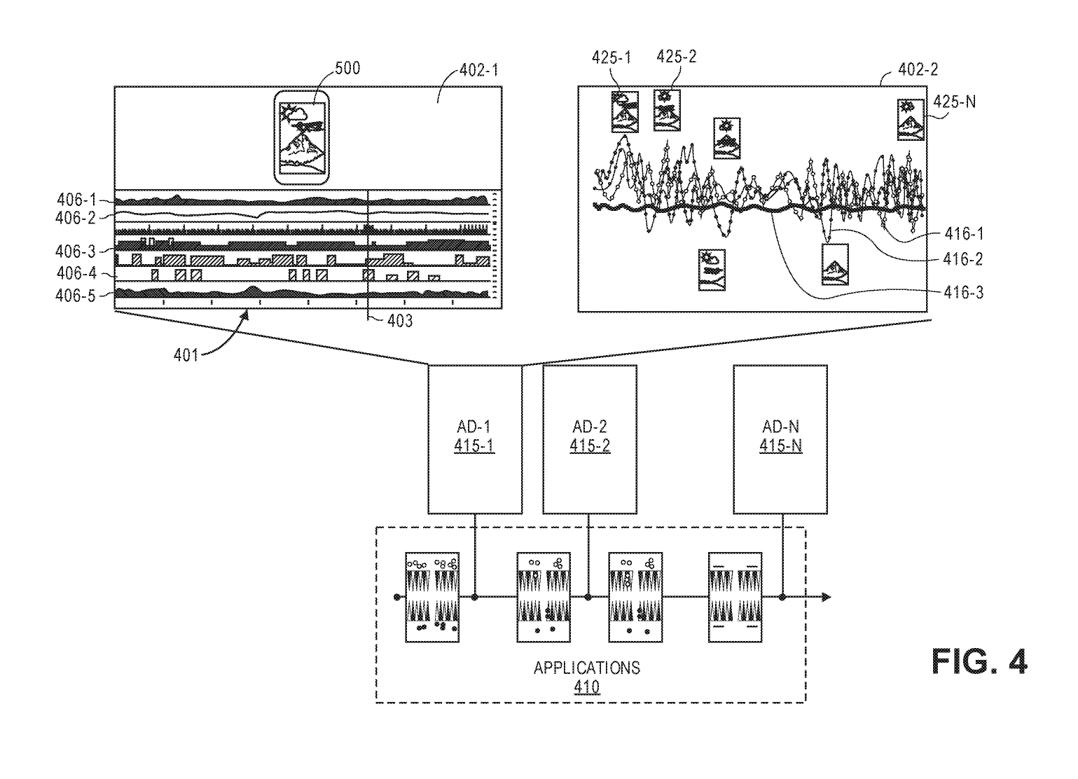

For example, FIG. 4 illustrates user collected sensor data, also referred to herein as response data, produced in response to the user 303 viewing the advertisement 315, in accordance with described implementations. In this example, the user interacts or plays an application 410 and is periodically presented with advertisements 415-1, 415-2-415-N at various ad placement positions within the advertisement. As will be appreciated, any number and/or type of advertisements may be presented to the user. As the user view the advertisements and/or interacts with the application, sensor data may be collected from the various sensors and time correlated with the presented advertisement or application. For example, sensor data visualization 402-1 provides a visual illustration of the sensor data collected as the advertisement 415-1 is presented to the user. For example, graph 406-1 is a visualization of the sensor data collected over time 401 from the EEG sensor 304-1, graph 406-2 is a visualization of the sensor data collected over time 401 from the device camera 304-2, graph 406-3 is a visualization of the sensor data collected over time 401 from the microphone 304-3, graph 406-4 is a visualization of the sensor data collected over time 401 from the GSR sensor 304-4, and graph 406-5 is a visualization of the sensor data collected over time 401 from the camera 304-5. In some implementations, sensor data may be collected before, during, and/or after the advertisement. For example, sensor data may be collected for a defined period of time before the advertisement to establish a baseline for the user and/or to determine environmental conditions before the advertisement is presented. Likewise, sensor data may be collected after completion of the advertisement to monitor long term influence of the advertisement on the user.

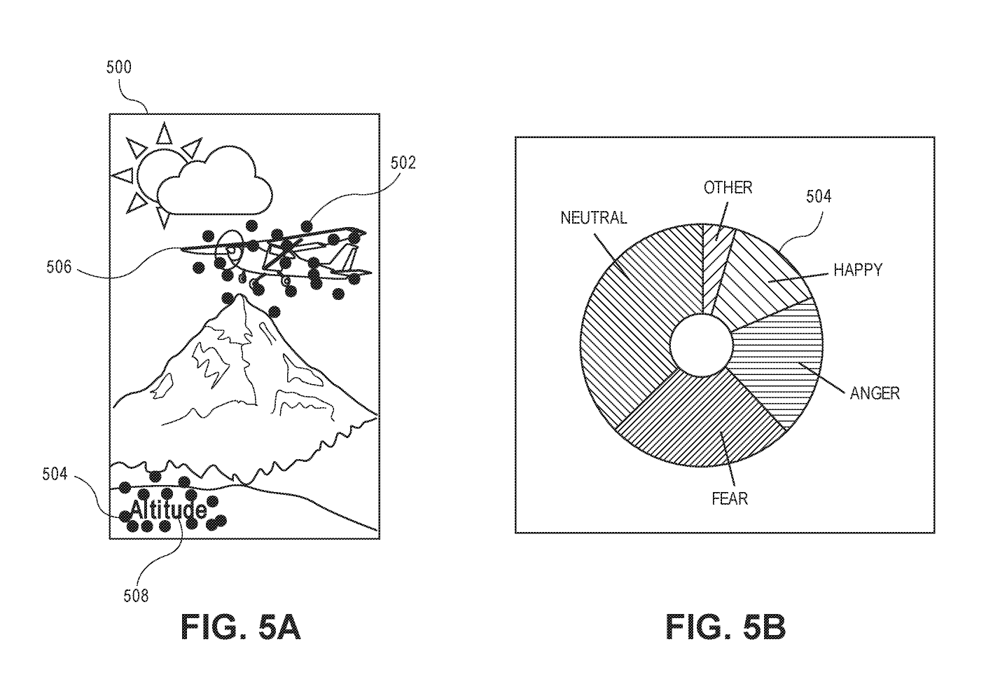

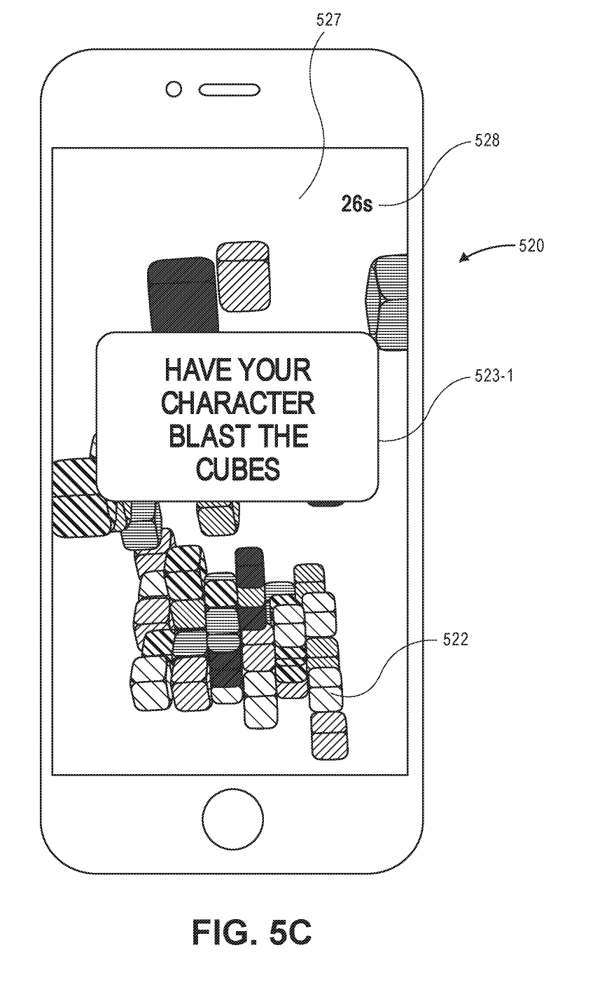

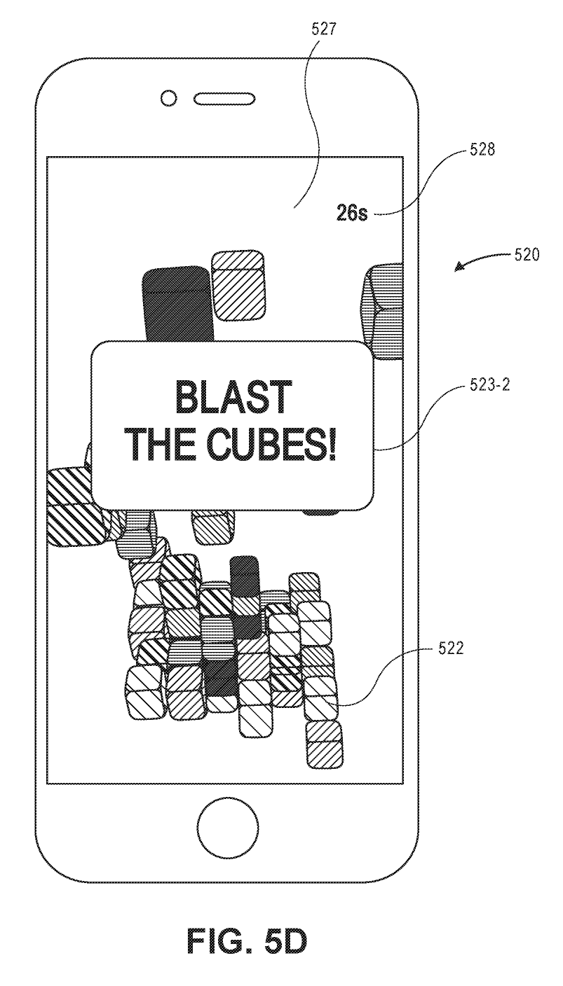

Based on the visualization 402-1 of the sensor data, particular points in time, such as point 403 may be identified in which the sensor data collectively indicates a particular response from the user. For example, at point 403 the sensor data 406-1, 406-2, 406-3, 406-4 indicate a change or peak in activity by the user at that point in time of the advertisement and it may be determined from that combination of sensor data that the user has presented a response of interest to the advertisement. Likewise, the presented segment 500 of the advertisement may be updated to correspond to a selected point in time, such as point 403. Referring briefly to FIG. 5A, illustrated is a detail view of the segment 500 of the advertisement corresponding to point 403. In this segment illustrated is an airplane 506 in the center of the advertisement and a brand name 508 presented in the lower corner of the advertisement. Overlaid on the segment 500 are visual identifiers or hot spots 502, 504, determined based on a processing of the sensor data (video) from the device camera 304-2 or the camera 304-5. The hot spots 502, 504 are indicative of a user gaze direction, determined from eye tracking, during this segment 500 of the advertisement. Based on this visualization, it may be further determined that, in this example, the presentation of both a main item, such as the airplane 506 and a secondary item, such as the brand name 508 may cause distraction to users viewing the advisement, as indicated by the variation in the eye tracking between the two presented items. Such information may be used alone, for example to refine an existing advertisement, and/or, as discussed below aggregated with other sensor data from other users viewing the advertisement as part of a machine learning system input to produce a model.

Returning to FIG. 4, visualization 402-2 presents another view of sensor data over time and corresponding to different segments of the advertisement 415-1. In this example, the different sensor data is visualized as line graphs 416-1, 416-2, and 416-3 and different segments 425-1, 425-2-425-N may be presented along with the visualization of the sensor data. In this example, rather than presenting the sensor data individually, one or more items of sensor data may be combined, and line graphs produced indicating a response that can be determined from a combination of that sensor data. For example, line graph 416-1 may be a combination of the sensor data indicative of system 1 responses to colors, sights, and sounds and line graph 416-2 may be a combination of sensor data indicative of system 2 responses to text, language, and logic.

FIG. 5B illustrates yet another example visualization 504 of user responses, such as neutral, happy, anger, feature, other, as determined from sensor data collected in a controlled environment as the user receives a presentation of an advertisement.

Collection of sensor data within a controlled environment reduces or holds approximately constant the influence of other variables (e.g., noise, temperature, lighting) that are external to the application so that a direct correlation between the user responses, as measured by the sensor data, and variables (color, text size, sound, etc.) of the presented advertisements can be measured.

FIG. 5C is a graphical illustration of an advertisement 520 that results in high cognitive load of a user, as indicated by sensor data from an EEG sensor. In comparison, FIG. 5D is a graphical illustration of the same advertisement 520 modified based on output from the machine learning system to reduce the amount of text presented to the user, resulting in reduced cognitive load and higher user engagement, in accordance with described implementations.

In the illustrated example, the advertisement 520 of FIG. 5C includes the text “Have Your Character Blast The Cubes” 523-1, which is determined by the described implementations to reduce user engagement and require high cognitive load by the user to process the information. In comparison, the same advertisement 520 of FIG. 5D with the modified text “Blast The Characters!” 532-2, produced based on a configuration file output from the trained machine learning system to simplify the text and increase the size of the font, results in higher user engagement and reduced cognitive load on the user viewing the advertisement.

In the illustrated examples, the client device presents on a display 527 of the device the advertisement 520. The advertisement, which in this example includes the cubes to be blasted, is presented on the display and may be interacted with by the user. Overlaid on the advertisement 520 is a call to action 523 instructing the user how to interact with the advertisement. The call to action 523-1 presented in FIG. 5C is long and wordy, resulting in higher cognitive load requirements of the viewing user, compared to the simplified call to action 523-2 presented in FIG. 5D, which results in higher user engagement.

As discussed herein, the described implementations are operable to dynamically generate a variety of advertisements for an ad campaign for presentation to a user that is accessing an application on a client device. While the above example illustrate advertisements that include modified text and font size, in other implementations other variables may be changed. For example, the colors of the advertisement, characters in the advertisement, duration of the advertisement, sounds included in the advertisement, font type, font treatment, text content, an object included in the advertisement, a video type, a character type, an animation, an interactive element (e.g., character, object), an interaction complexity, an image, a haptic output, a brightness, a contrast, etc., may be modified or adjusted based on a configuration file produced from the trained machine learning system.

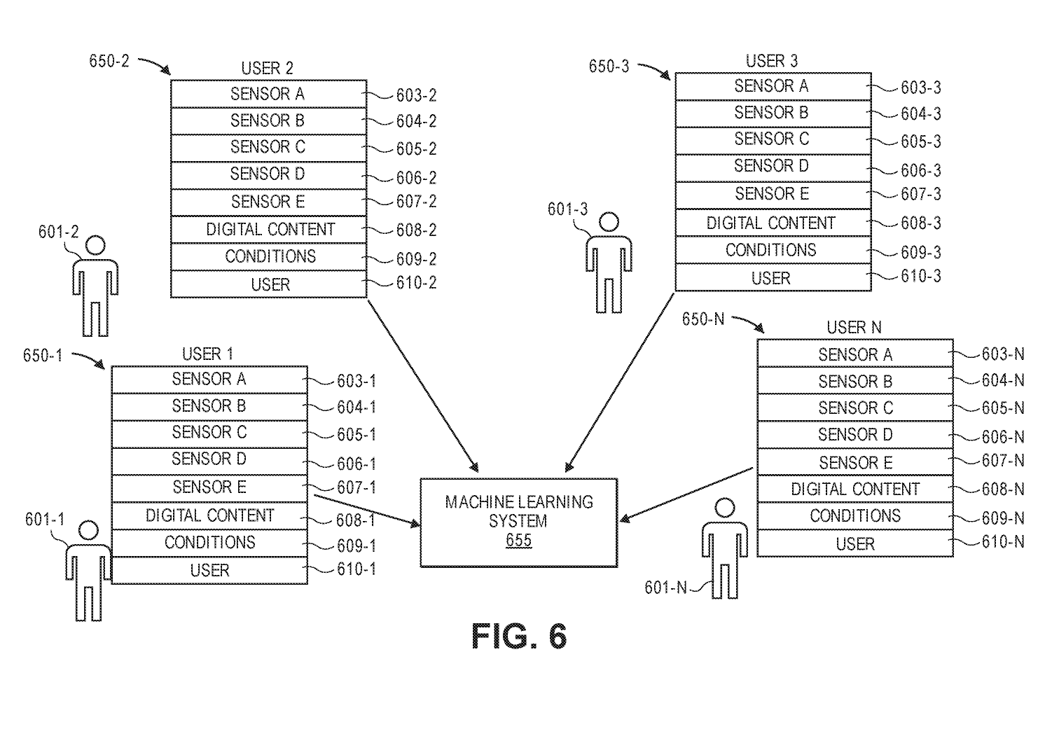

FIG. 6 illustrates a plurality of users 601(1), 601(2), 601(3), -601(N) that interact with applications and/or view digital content, such as advertisements, presented on a respective user device and have corresponding user profiles 610-1, 610-2, 610-3, -610-N. As the users 601 interact with digital content presented to the users, actual sensor data is collected and included as part of response profile 650-1, 650-2, 650-3, and 650-N. In addition, other data, generally referred to herein as conditions 609, may also be included in the response profile as actual conditions at the time the digital content is presented to the user. For example, the device profile (e.g., device type, orientation, connectivity, etc.), and/or environmental conditions (e.g., device/user location, time of day, day of week, temperature, lighting, ambient noise) may be determined and included in each response profile 650.

For example, when user 1 601-1 is accessing an application and is presented with digital content, the resulting response profile may include sensor A data 603-1, sensor B data 604-1, sensor C data 605-1, sensor D data 606-1, sensor E data 607-1, and indication of the digital content 608-1 that was presented, the conditions 609-1 under which the user 601-1 viewed the digital content, and the user profile 610-1 corresponding to the user 601-1. In addition, an actual user response and/or engagement may likewise be determined and stored as part of the response profile 650-1 for the user 601-1.

In a similar manner, when user 2 601-2 is accessing an application and is presented with digital content, the resulting response profile may include sensor A data 603-2, sensor B data 604-2, sensor C data 605-2, sensor D data 606-2, sensor E data 607-2, and indication of the advertisement 608-2 that was presented, the conditions 609-2 under which the user 601-2 viewed the advertisement, and the user profile 610-2 corresponding to the user 601-2. In addition, an actual user response or engagement may likewise be determined and stored as part of the response profile 650-2 for the user 601-2. In a similar manner, when user 3 601-3 is accessing an application and is presented with the digital content, the resulting response profile may include sensor A data 603-3, sensor B data 604-3, sensor C data 605-3, sensor D data 606-3, sensor E data 607-3, and indication of the advertisement 608-3 that was presented, the conditions 609-3 under which the user 601-3 viewed the advertisement, and the user profile 610-3 corresponding to the user 601-3. In addition, an actual user response or engagement may likewise be determined and stored as part of the response profile 650-3 for the user 601-3. In a similar manner, when user N 601-N is accessing an application and is presented with digital content, the resulting response profile may include sensor A data 603-N, sensor B data 604-N, sensor C data 605-N, sensor D data 606-N, sensor E data 607-N, and indication of the advertisement 608-N that was presented, the conditions 609-N under which the user 601-N viewed the advertisement, and the user profile 610-N corresponding to the user 601-N. In addition, an actual user response or engagement may likewise be determined and stored as part of the response profile 650-N for the user 601-N.

As will be appreciated, each of the users 601-1, 601-2, 601-3 through 601-N may be in a controlled environment, in which case the conditions 609 are known and controlled, or an uncontrolled environment, in which case the conditions may be measured by one or more sensors or otherwise determined (e.g., from a third-party service).

As the response profiles are generated with actual information corresponding to digital content, the response profiles are provided to a machine learning system 655 as training inputs.

Because users vary in their preferences, interests, application use patterns, behaviors, etc., and those things further vary based on other conditions (e.g., time of day, day of week, time of year, location, weather, etc.), actual user responses (e.g., system 1 and system 2) and resultant engagements exhibited by users in response to different digital content with different variables (e.g., color, sound, text size, objects) will likewise vary. As a particular example, if an advertisement, such as the advertisement discussed above with respect to FIG. 4 and FIGS. 5A-5B is presented to three different users at the same advertisement placement position within the same application and within the same controlled environment, the responses (system 1 and system 2) from each user may vary.

In accordance with the present disclosure, the response profile, such as the collected sensor data discussed above with respect to FIG. 4 and the other aspects of a response profile discussed with respect to FIG. 6 may be provided as inputs to a machine learning system 655, either in real time or in near-real time, as the data is collected in response to presented digital content. In addition to the training inputs, in some implementations, actual user engagements by other similar users in response to the presented digital content may be provided to the machine learning system.

The machine learning system 655 may be fully trained using a substantial corpus of response profiles, digital content, and/or engagement with that digital content to develop models for different user types under different conditions. For example, a user type may include users having similar user profiles and that produce similar responses to segments or forms of digital content presented under the same or similar conditions. Such types of users may be correlated and associated as a user type by the machine learning system 655 and utilized as representative of users that are similar. Likewise, after the machine learning system 655 has been trained, and the models developed, the machine learning system may be provided with a user profile, current conditions, application information, digital content placement position information, and a device profile, and the machine learning system will generate a model and corresponding engagement prediction that is to be used to produce or select digital content, such as an advertisement, to present to the user.

Training of the machine learning system may include thousands or millions of response profiles and corresponding conditions, digital content (e.g., advertisements), users, etc., under controlled and/or uncontrolled conditions.

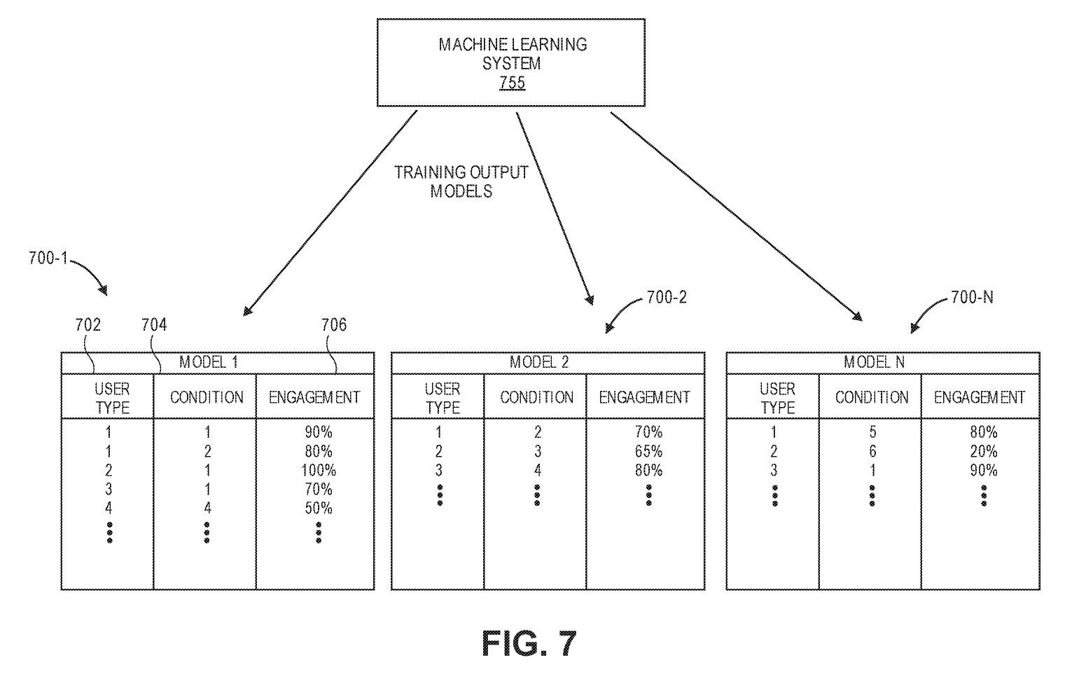

Referring to FIG. 7 illustrated are example models 700 that may be output by the machine learning system 755 in response to receiving training inputs. For example, the training inputs, as discussed above with respect to FIG. 6, may include multiple different response profiles for multiple different items of digital content and users under controlled and/or uncontrolled conditions. In some implementations, the machine learning system may develop models for different groups of users (i.e., user types) and/or for varying conditions, such as different applications, different digital content placements within an application, time, day, weather, location, etc.

The models 706 produced by the machine learning system may classify user types and conditions into different models, such as model 1 700-1, model 2 700-2, -model N 700-N. Each model may be associated with different user types and/or varying conditions and producing similar likelihood of engagement under those conditions. In some implementations, a model may be associated with or representative of hundreds, thousands, or millions different user types and condition pairs. Likewise, in some implementations, a response profile may correspond to one and only one model. In other implementations, a response profile may correspond to different models, for example, depending on the desired engagement, desired response, and/or the conditions.

Included in each model is an indication of the user type (e.g., demographics, age range, device types, etc.) corresponding to the model and representative of the response profiles utilized to develop the model 700. For example, model 700-1 includes user types 702. Likewise, each model may indicate one or more conditions 704 to form a user type and condition pair, and an engagement prediction 706 for the user type condition pair when digital content, such as an advertisement, corresponding to the model is presented to a user of that user type under similar conditions. As discussed above, the conditions may indicate, for example, any one or more of weather, location, time of day, day of week, week of month, month of year, activity, application, digital content placement position, etc.

The models also include predicted engagement values 706 for each combination of user type 702 and condition 704. The predicted engagement indicates the probability of a user engagement (e.g., click-through, purchase, interaction, etc.) in response to a digital content presented to a user similar to the determined user type with the other conditions being present.

Those of ordinary skill in the pertinent arts will recognize that any type or form of machine learning system (e.g., hardware and/or software components or modules) may be utilized in accordance with the present disclosure. For example, one or more machine learning algorithms or techniques, including but not limited to nearest neighbor methods or analyses, artificial neural networks, conditional random fields, factorization methods or techniques, K-means clustering analyses or techniques, similarity measures such as log likelihood similarities or cosine similarities, latent Dirichlet allocations or other topic models, or latent semantic analyses may be utilized alone or in combination to develop user models, predict likelihood of user engagement and user responses to digital content generated according to variables specified in a model.

In some implementations, a machine learning system may identify not only a predicted engagement but also a confidence interval, confidence level or other measure or metric of a probability or likelihood that the predicted engagement will be exhibited by a user that is similar to the user type in response to particular digital content generated according to the model and presented to the user under particular conditions. Where the machine learning system is trained using a sufficiently large corpus of response profiles, user information, conditions, recommended digital content, actual user engagement, etc., the confidence interval associated with the predicted engagement may be substantially high.

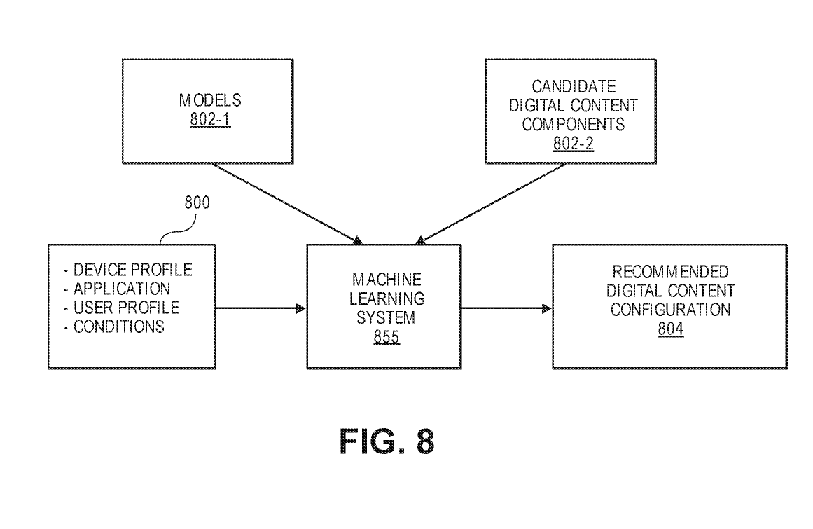

Referring to FIG. 8, illustrated is the trained machine learning system 855 receiving inputs 800 of a user profile for user A, device profile, conditions at the location of the device, and application information indicating an application through which digital content is to be presented. The conditions may be provided by the device and include, for example, location of the device, orientation of the device, altitude, temperature, etc. Alternatively, or in addition thereto, conditions may be obtained from a third-party service. For example, the machine learning system 855 may receive location information for the device and then obtain condition information (e.g., weather) for that location from a third party. In addition to receiving inputs 800, the machine learning system may determine or receive candidate digital item components, such as advertisement components, that have been provided by a third party, such as an advertiser, for generating an item of digital content. The digital item components may include or identify different components that may be included in an item of digital content (e.g., an advertisement), such as different font sizes, different sounds and/or amplitude of sounds, different colors, different images, different videos, different objects, different haptics, different durations, different image types, different animations, different interactive elements and/or the complexity of the interaction, different haptic outputs, brightness, contrast, etc.

In addition, a model 802-1 may be determined based on a combination of one or more of the inputs 800 and the candidate advertising components 802-2. For example, a model that includes a user type and condition pair that most similarly matches the user profile and conditions of the inputs may be determined and selected as the model 802-1.

Utilizing the model, the inputs 800, and the candidate digital item components, the machine leaning system determines a recommended digital content configuration 804 for use in generating digital content for presentation to the user. In response, a recommendation system, such as an advertisement recommendation system, may select or generate digital content corresponding to the recommended digital item configuration and send the recommended digital content to a user device in use by the user for presentation to the user.

While the above examples discuss the use of a machine learning system 855 for generating a recommended digital content configuration, such as a recommended advertisement configuration, based on candidate digital item components, in other implementations, the machine learning system may generate digital content variables, such as advertisement variables, that, if incorporated into the creation of a digital content will produce a desired response and predicted engagement. For example, the machine learning system may receive the inputs 800 and an indication of one or more desired responses and select a model that most closely corresponds to the inputs 800 and desired response. Based on the model, digital content variables (e.g., color, speed of video, size and/or type of images, animations, characters, haptics, font, sound, objects, etc.) may be indicated in the configuration file as recommended variables for use in generating digital content that will produce the desired response from a user of a particular user type and under the input conditions.

For example, the configuration file for a particular user or group of user may indicate a model for the user or group of users, digital content variables, such as but not limited to, colors or color contrasts between colors, brightness, font size, font type, font treatment, amount of text to include in the advertisement, text placement, sounds, haptics, duration, inaction, interaction complexity, contrast, etc., that may be utilized in dynamically generating an advertisement and/or selecting an existing advertisement, etc.

In still another example, the machine learning system may receive the inputs 800, determine an appropriate model based on the inputs 800, and select an existing item of digital content, such as an advertisement, that most closely corresponds to the determined model, user type and conditions to elicit a desired response from a user of the user type when the selected digital content is presented to the user.

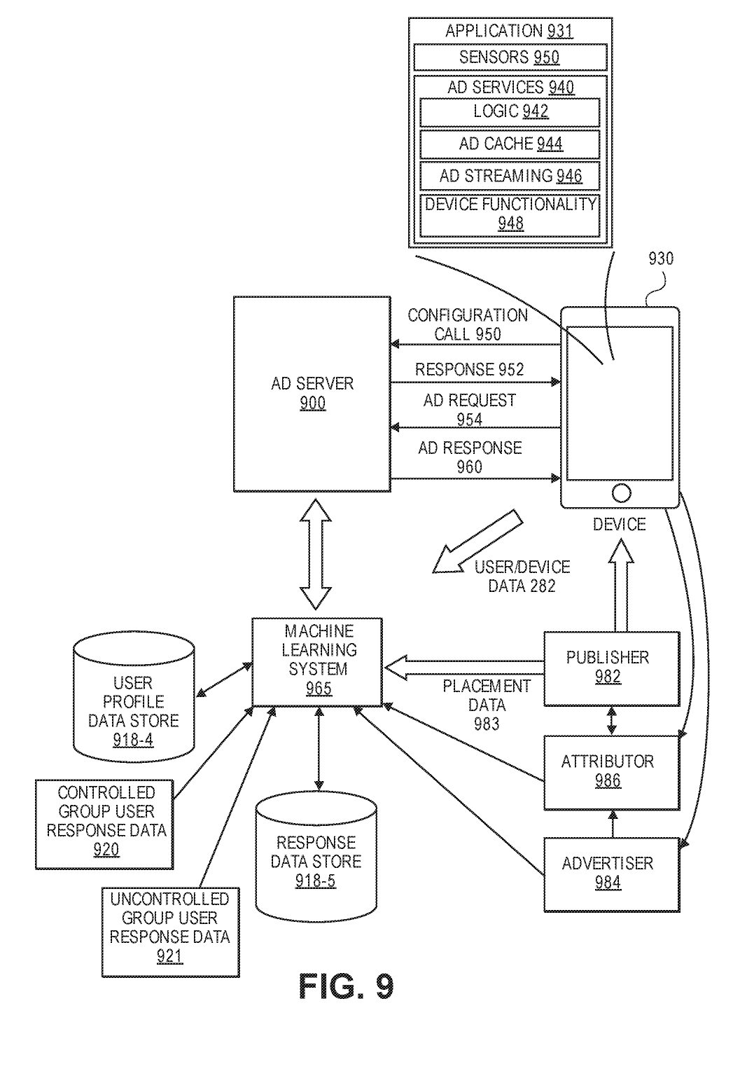

FIG. 9 is a block diagram illustrating the exchange of information between an ad server system 900, a user device 930, publishers 982, an advertiser 984, and an attributor 986, in accordance with described implementations. As with other examples discussed herein, while this example is described with respect to advertisements, it will be appreciated that the described implementations are equally applicable to other forms of digital content.

In the example illustrated in FIG. 9, a user device 930 (e.g., source device, client device, mobile phone, tablet device, laptop, computer, wearable, connected or hybrid television (TV), IPTV, Internet TV, Web TV, smart TV, etc.) initiates a software application (e.g., a mobile application, a mobile web browser application, a web based application, non-web browser application, etc.). For example, a user may select one of several software applications 931 installed on the user device. The advertising services software 940 operating on the user device 930, is also initiated upon the initiation of one of the software applications 931. Likewise, in some implementations, one or more sensors 950 of the device and/or in communication with the device (e.g., sensors of a wearable device worn by the user that is in communication with the device 930) may also be initiated and used to collected sensor data relating to the user and/or the environment.

The advertising services software 940 may be associated with or embedded with the software application. The advertising services software 940 may include or be associated with logic 942 (e.g., communication logic for communications such as an ad request), an ad cache store 944 for storing one or more of the ads provided by the ad server system 900 to the user device 930, ad streaming functionality 946 for receiving, optionally storing, and playing ads streamed from the ad server system 900, and device functionality 948 for determining device and connection capabilities (e.g., type of connection (e.g., 4G LTE, 3G, Wi-Fi, WiMAX, etc.), bandwidth of connection, location of device, type of device, display characteristics (e.g., pixel density, color depth, etc.). The initiated software application(s) or advertising services software 940 may have an ad play event for displaying or playing an ad on the display of the device 930. At operation, the ad services 940 generates and sends to the ad server system 900 a configuration call request 950. The configuration call may include information about the user accessing the software application, user device information, location, sensor data collected from the sensors 950 and/or other information collected by the logic 942.

The ad server system, in response to receiving a configuration call 950, determines options for ad play events corresponding to the software application being accessed on the user device 930 to be utilized to present advertisements to the user. The options may include, but are not limited to, options for obtaining an advertisement to present on the user device in response to an ad play event, ad placement positions that are to be utilized within the software application to present advertisements to the user, etc. In one implementation, a first option may include playing at least one ad that is cached in the ad cache memory 944 of the user device 930 during the ad play event. A second option may include generating or selecting an ad that corresponds to a determined model, as discussed above. If the ad server system generates and sends another ad in a timely manner (e.g., in time for a predicted ad play event at a selected ad placement position, within a time period set by the at least one configuration file) then the provided ad will be presented during the predicted ad play event at the ad placement position within the software application 931. Another option may include streaming at least one ad to be played during the predicted ad play event to the device 930. The configuration file can be altered by the ad server system 900 or the user device 930 without affected the advertising services software 940.

As a user is accessing the software application 931, an ad placement position at a future location within the software application that has been indicated as an ad placement position to be utilized is determined and the ad services 940 sends an ad request 954 to the ad server system 900 requesting an advertisement to present at that ad placement position. The ad server system 900, upon receiving the ad request 954, utilizes the information from the configuration file and optionally any additional information included in the ad request 954, information from the attributer 986, and/or information from the advertiser 984 to determine a model and dynamically generate and send an advertisement to the user device 930 for presentation at the ad placement position in response to the ad play event. Dynamic generation of an ad is discussed above.

The ad request 954 may include different types of information, including, but not limited to, publisher settings (e.g., a publisher of the selected software application), an application identifier identifying the software application 931, ad placement position information for timing placement of an ad in-app, user profile information, user device characteristics (e.g., device id, OS type, network connection for user device, whether user device is mobile device, volume, screen size and orientation, language setting, etc.), environmental conditions (e.g., geographical data, location data, motion data, such as from an accelerometer or gyroscope of the user device, weather), language, time, application settings, demographic data for the user of the device, access duration data (e.g., how long a user has been using the selected application), and cache information. The ad server system 900 processes the ad request 954, and optionally other information, as discussed herein, to determine an appropriate model and either dynamically generate or select and existing advertisement that is sent to the user device as the ad response 960 for presentation at the ad placement position.

Attributers 986 may have software (e.g., an SDK of the publisher of the application) installed on the user's device and obtain third party user data from the user device. This user data may include, but is not limited to, a user's interaction and engagement with the software application, a length of time that the application is installed, an amount of purchases from the application, buying patterns in terms of which products or services are purchased and when these products or services are purchased, engagement with advertisements presented in applications, access duration by a user of an application, etc. As the attributor collects data from the user device, the collected data may be provided to a machine learning system 965, which may be included in the ad server system or separate from the ad server system. In some implementations, data may also or alternatively be provided directly from the user device to the machine learning system 965.

In some implementations, advertisers 984, the ad server system 900, controlled group response data 920 and/or uncontrolled group response data may also provide data to the machine learning system 965. For example, the ad server system 900 may provide information indicating candidate advertisement components, and/or ad placement positions used for generating and presenting ads to a particular user in a particular application, etc. Likewise, in some implementations, publishers 982 may also provide information to the machine learning system, including, but not limited to, application information (e.g., type and/or content of the application), candidate ad placement positions, etc. Still further, the machine learning system may obtain data from a user profile data store 918-4 corresponding to a user associated with the device 930 and/or obtain information from the response data store 918-5. The user profile information maintained in the user profile data store may include any information about the user associated with the device 930. Response data included in the response data store may include historical response data corresponding to the user associated with the device 930 and/or response data corresponding to other users that are determined to be similar to the user interacting with the device 930.

The machine learning system 965, as discussed above, upon receiving information from the ad server system 900, device 930, publisher 982, attributors 986, advertisers 984, controlled groups 920 and/or uncontrolled groups 921, may generate models representative of advertisement variables that may be used to produce advertisements to elicit an intended user response and an expected engagement. The models may be generated based on actual sensor data collected under controlled and/or uncontrolled conditions and indicate advertising variables that, if incorporated into an advertisement, will produce a desired response from the user when viewed under the provided conditions. Machine learning and the generation of models is discussed above with respect to FIGS. 6-8.

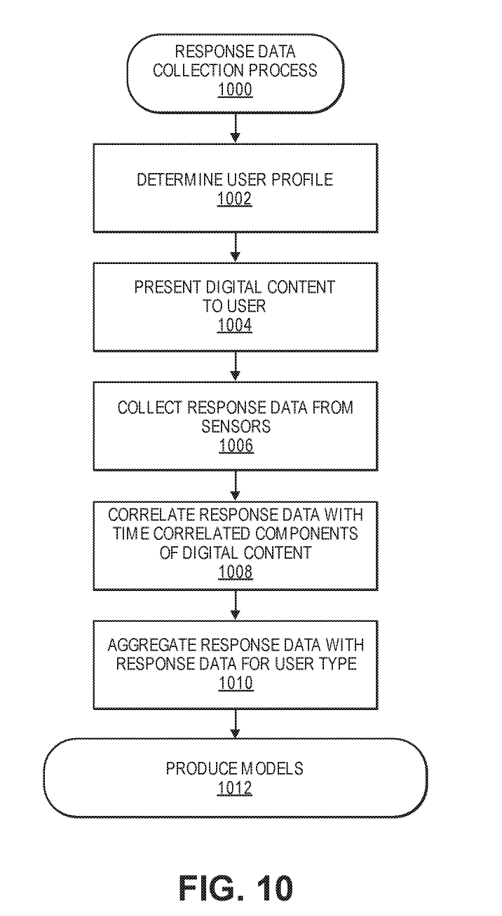

FIG. 10 is an example response data collection process 1000, in accordance with described implementations. The example process 1000 may be performed in a controlled environment or an uncontrolled environment. The example process 1000 begins by determining a user profile of a user for which response data is to be collected, as in 1002. Any variety of techniques may be used to determine a user profile. For example, the user may provide identifying information, such as a user name and password, biometrics, visual identifier, etc. In other implementations, if the user does not have a user profile or is not known to the system, the user may provide user information necessary to produce a user profile.

To collect response data, digital content, such as an advertisement, is presented to the user, as in 1004. Presentation of a digital content may be an audible presentation, video presentation, tactile presentation, or any combination thereof. For example, one component of an advertisement (digital content) may be visually presented on a touch-based display of a portable device, another component of the advertisement may be audibly presented using a speaker of the portable device, and a third component of the advertisement may be haptically presented using an actuator of the portable device.

As the digital content is presented to the user, response data is collected by one or more sensors (e.g., EEG, fMRI, GSR, microphone, camera, accelerometer) as representative of the user's response to the presentation of the digital content, as in 1006. As discussed above, the collected response data is time correlated with the presentation of the digital content such that components of the digital content relate to points in time, as in 1008. As illustrated above, the time correlated digital content and response data may be presented visually to indicate peaks and valleys in the collected data and corresponding to different segments of the digital content.

In addition, response data from the user is aggregated with response data from other users viewing the same and/or different digital content, as in 1010. The aggregated response data may be utilized to determine similar variables among the presentations that produce similar responses from similar users and/or dis-similar users. For example, some variables (color, sound, images, objects) may produce a specific response for only certain user types while other variables may produce a similar response from all users, regardless of type. By aggregating response data from a large corpus of users the different responses from similar or dis-similar users may be identified and correlated with the respective variables.

Finally, based on the aggregated response data and the determined correlations, utilizing a machine learning system, as discussed above, one or more models are produced or updated based on the information, as in 1012. Generation of models based on machine learning is discussed above.

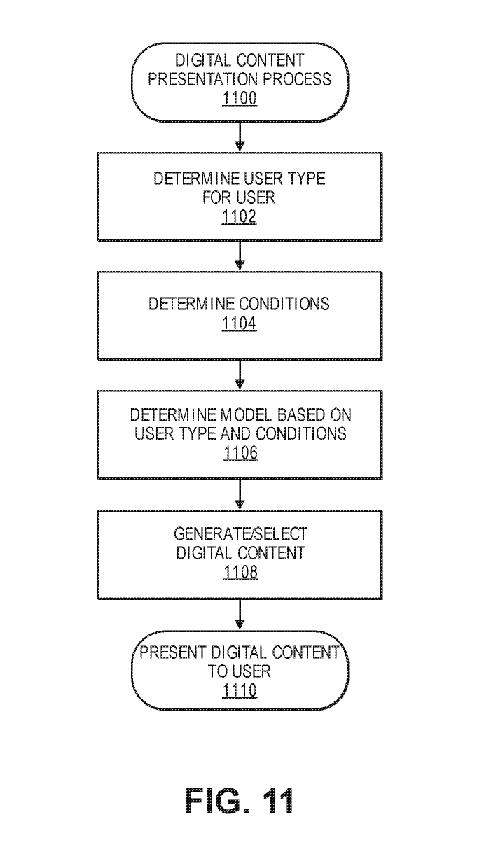

FIG. 11 is an example digital content presentation process 1100, in accordance with described implementations. The example process 1100 begins by determining a user type for a user to which digital content, such as an advertisement, is to be presented, as in 1102. The user type may be determined, for example, based on a user profile associated with the user and/or in response to a user input or other user information.

In addition to determining the user type, one or more conditions are determined, as in 1104. As discussed above, conditions may include, but are not limited to, an application in which the digital content is to be presented, a device type, device orientation, weather, location, time of day, day of week, week of month, month of year, etc.

Based on the user type and the conditions, a model generated by the machine learning system, discussed above, is selected that is most likely to produce a desired response and engagement, as in 1106. For example, the models may be maintained in a model data store and queried based on the user type and conditions to determine a model that best matches the user type and condition pair.

Based on the determined model, digital content is generated or selected that includes variables indicated in the model that, when presented to the user, will produce the desired response and increase the likelihood of engagement with the digital content, 1108. Finally, the generated or selected digital content is presented to the user, as in 1110.

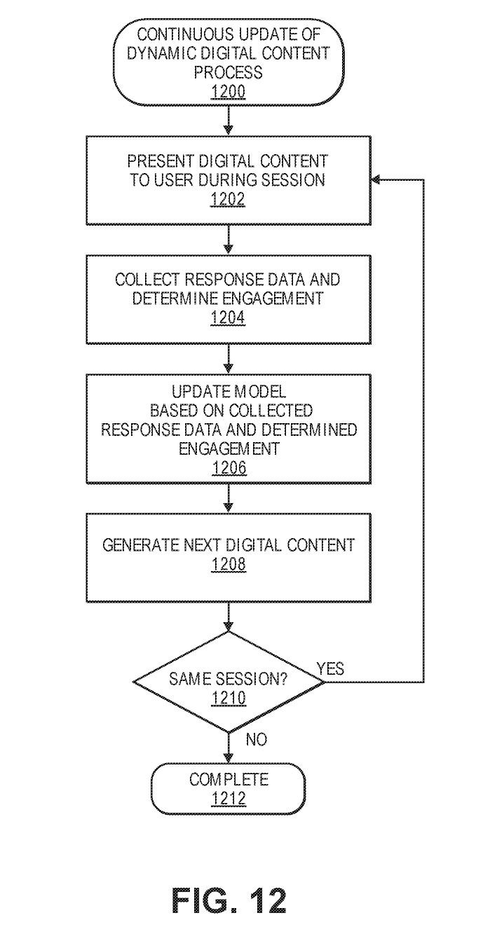

FIG. 12 is an example continuous update of dynamic digital content process 1200, in accordance with described implementations. The example process 1200 beings with the presentation of digital content to a user during a session, as in 1202. A session may be any discrete amount of time during which a user is accessing a device through which digital content is to be presented. For example, the session may be a period of time that the user is accessing an application through which advertisements (digital content) are to be presented. As another example, the session may be 8:00-14:00, or any other period of time.

As the digital content is presented, response data is collected using one or more sensors and user engagement with the digital content is also determined, as in 1204. As discussed above, response data may include sensor data from any of a variety of sensors that are on the user device, in communication with the device, or for which sensor data is otherwise accessible. For example, if the presentation is in a controlled environment, some or all of the response data may be collected via sensors that are independent of the device through which the digital content is being presented.

The response data and determined engagement may then be used to update the model for that user during that session, as in 1206. In some implementations, the update may be specific to the user and/or the session. For example, the response data received from the user during the session may be influenced by the user's current mood or attitude. In such an example, the response data collected during the session may receive a greater weight and be utilized to determine a model that is closely correlated with the user based on the current response data. In other implementations, the current response data may be persistent and utilized to update the models for user types that are similar to the user.

Based on the updated model, a next item of digital content is generated based on the updated model such that the digital content includes variables that are most likely to produce a desired response from the user and resulting engagement with the digital content, as in 1208. In addition, a determination may be made as to whether the user is still in the same session, as in 1210. For example, if the session corresponds to the user's application access or period of time, it may be determined whether the same session exists for the user.

If it is determined that the same session exists, the example process 1200 returns to block 1202 and continues by presenting the next digital content to the user, as in 1210. However, if it is determined that the session has ended, the example process 1200 completes, as in 1212. The example process 1200 may be performed continuously as users interact with applications and receive digital content, such as advertisements, so that digital content can be continually revised to best match the current mood of the user for which the digital content is to be presented.

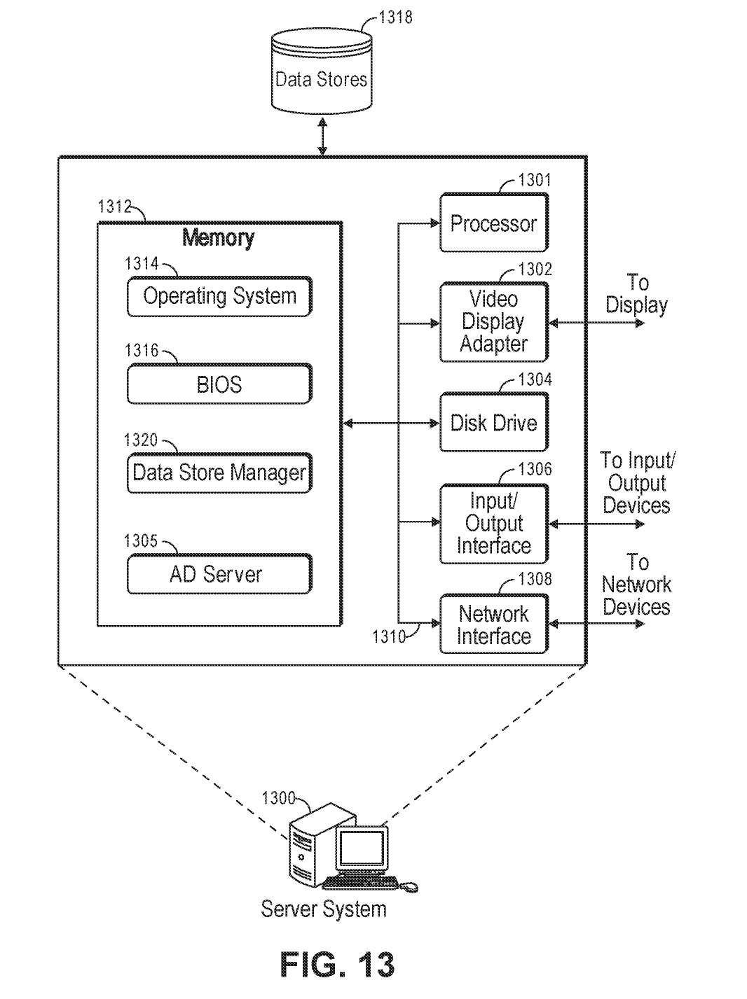

FIG. 13 is a pictorial diagram of an illustrative implementation of a server system 1300, such as a remote computing resource, that may be used with one or more of the implementations described herein. The server system 1300 may include a processor 1301, such as one or more redundant processors, a video display adapter 1302, a disk drive 1304, an input/output interface 1306, a network interface 1308, and a memory 1312. The processor 1301, the video display adapter 1302, the disk drive 1304, the input/output interface 1306, the network interface 1308, and the memory 1312 may be communicatively coupled to each other by a communication bus 1310.

The video display adapter 1302 provides display signals to a local display permitting an operator of the server system 1300 to monitor and configure operation of the server system 1300. The input/output interface 1306 likewise communicates with external input/output devices, such as a mouse, keyboard, scanner, or other input and output devices that can be operated by an operator of the server system 1300. The network interface 1308 includes hardware, software, or any combination thereof, to communicate with other computing devices. For example, the network interface 1308 may be configured to provide communications between the server system 1300 and other computing devices, such as the user device 1100.

The memory 1312 generally comprises random access memory (RAM), read-only memory (ROM), flash memory, and/or other volatile or permanent memory. The memory 1312 is shown storing an operating system 1314 for controlling the operation of the server system 1300. A binary input/output system (BIOS) 1316 for controlling the low-level operation of the server system 1300 is also stored in the memory 1312.

The memory 1312 additionally stores program code and data for providing network services that allow user devices and external sources to exchange information and data files with the server system 1300. The memory also stores a data store manager application 1320 to facilitate data exchange and mapping between the data store 1318, ad server system 1305, user devices, external sources, etc. While this describes interaction with an ad server system 1305, the describe implementations, and the described example server system 1300, may likewise be utilized with any form of digital content server.

As used herein, the term “data store” refers to any device or combination of devices capable of storing, accessing and retrieving data, which may include any combination and number of data servers, databases, data storage devices and data storage media, in any standard, distributed or clustered environment. The server system 1300 can include any appropriate hardware and software for integrating with the data store 1318 as needed to execute aspects of one or more applications for the user device 1100, the external sources and/or the ad server system 1305. The server system 1300 provides access control services in cooperation with the data store 1318 and is able to generate digital content, such as advertisements.

The data store 1318 can include several separate data tables, databases or other data storage mechanisms and media for storing data relating to a particular aspect. For example, the data store 1318 illustrated includes advertisements, digital content items for advertisements, models, user profiles, etc.

It should be understood that there can be many other aspects that may be stored in the data store 1318, which can be stored in any of the above listed mechanisms as appropriate or in additional mechanisms of any of the data stores. The data store 1318 may be operable, through logic associated therewith, to receive instructions from the server system 1300 and obtain, update or otherwise process data in response thereto.

The memory 1312 may also include the ad server system 1305. The ad server system 1305 may be executable by the processor 1301 to implement one or more of the functions of the server system 1300. In one implementation, the ad server system 1305 may represent instructions embodied in one or more software programs stored in the memory 1312. In another implementation, the ad server system 1305 can represent hardware, software instructions, or a combination thereof. The ad server system 1305 may perform some or all of the implementations discussed herein, alone or in combination with other devices.

The server system 1300, in one implementation, is a distributed environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 13. Thus, the depiction in FIG. 13 should be taken as being illustrative in nature and not limiting to the scope of the disclosure.

The concepts disclosed herein may be applied within a number of different devices and computer systems, including, for example, general-purpose computing systems, and distributed computing environments.

The above aspects of the present disclosure are meant to be illustrative. They were chosen to explain the principles and application of the disclosure and are not intended to be exhaustive or to limit the disclosure. Many modifications and variations of the disclosed aspects may be apparent to those of skill in the art. Persons having ordinary skill in the art should recognize that components and process steps described herein may be interchangeable with other components or steps, or combinations of components or steps, and still achieve the benefits and advantages of the present disclosure. Moreover, it should be apparent to one skilled in the art that the disclosure may be practiced without some or all of the specific details and steps disclosed herein.

Aspects of the disclosed system may be implemented as a computer method or as an article of manufacture such as a memory device or non-transitory computer readable storage medium. The computer readable storage medium may be readable by a computer and may comprise instructions for causing a computer or other device to perform processes described in the present disclosure. The computer readable storage media may be implemented by a volatile computer memory, non-volatile computer memory, hard drive, solid-state memory, flash drive, removable disk and/or other media. In addition, components of one or more of the modules and engines may be implemented in firmware or hardware.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

Language of degree used herein, such as the terms “about,” “approximately,” “generally,” “nearly” or “substantially” as used herein, represent a value, amount, or characteristic close to the stated value, amount, or characteristic that still performs a desired function or achieves a desired result. For example, the terms “about,” “approximately,” “generally,” “nearly” or “substantially” may refer to an amount that is within less than 10% of, within less than 5% of, within less than 1% of, within less than 0.1% of, and within less than 0.01% of the stated amount.

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A computer implemented method, comprising:
collecting a plurality of response data for a plurality of users of a first type in a controlled group as each of the plurality of users receive a presentation of digital content, wherein the response data includes at least one of an electroencephalography (“EEG”) data, a functional magnetic resonance imaging (“fMRI”) data, a galvanic skin response (“GSR”) data, a heart rate data, a body temperature data, an eye tracking data, a face tracking data, or a head tracking data;
time correlating the plurality of response data with a plurality of variables of the digital content;
determining, based at least in part on the time correlation, an expected user response to each of the plurality of variables related to the digital content; and
producing an advertisement based at least in part on the expected user response to each of the plurality of variables such that the advertisement is created to elicit a desired response from a user of the first type when presented to the user.

2. The computer implemented method of claim 1, further comprising:
producing, based at least in part on the response data and the plurality of variables a model for users of the first type.

3. The computer implemented method of claim 2, further comprising:
determining a user type of a user that is to receive the advertisement;
determining, based at least in part on the user type, the model; and
wherein producing the advertisement is based at least in part on the model.

4. The computer implemented method of claim 2, wherein the model indicates at least one variable that may be adjusted in the advertisement to produce a desired response from users of the first type.

5. The computer implemented method of claim 4, wherein the variable is at least one of a font size, a font type, a font treatment, a text content, a color, a duration, a sound, an object, a video type, a character type, an animation, an interactive element, an interaction complexity, an image, a haptic output, a brightness, or a contrast.

6. A system, comprising:
one or more processors; and
a memory storing program instructions that when executed by the one or more processors cause the one or more processors to at least:
produce a first item of digital content according to a model output by a machine learning system for a first user of a first type, wherein the model indicates at least one variable to be adjusted in the first item of digital content to cause an expected response from users of the first type;
cause the first item of digital content to be presented to the first user of the first type;
collect response data indicative of a response presented by the first user in response to the first item of digital content presented to the first user; and
update the model based at least in part on the response data to produce an updated model.

7. The system of claim 6, wherein the program instructions, when executed by the one or more processors, further cause the one or more processors to at least:
produce a second item of digital content according to the updated model output for a second user of a second type, wherein the second item of digital content is a variation of the first item of digital content, varied according to the updated model; and
cause the second item of digital content to be presented to the second user of the first type.

8. The system of claim 6, wherein the program instructions, when executed by the one or more processors, further cause the one or more processors to at least:
determine at least one condition corresponding to the first user of the first type;
provide the first type of the first user and the at least one condition to a machine learning system; and
receive, from the machine learning system, the model, wherein the model is based at least in part on the first type and the condition.

9. The system of claim 8, wherein the program instructions, when executed by the one or more processors, further cause the one or more processors to at least:
determine a desired response from the first user; and
wherein the model is further produced based at least in part on the first type, the condition, and the desired response.

10. The system of claim 6, wherein:
the first user is in a controlled environment; and
the response data is collected using one or more sensors within the controlled environment.

11. The system of claim 10, wherein the one or more sensors include one or more of:
an electroencephalography (“EEG”) sensor, a functional magnetic resonance imaging (“fMRI”) sensor, a galvanic skin response (“GSR”) sensor, a heart rate sensor, a body temperature sensor, an eye tracking sensor, a face tracking sensor, a head tracking sensor, a camera, a microphone, a pressure sensor, an accelerometer, or a gyroscope.

12. The system of claim 6, wherein the response is at least one of a primal response to the first item or a secondary response to the first item.

13. The system of claim 6, wherein the model is produced based on a plurality of response data collected from a plurality of users of the first type.

14. A method, comprising:
for each of a plurality of users:
presenting digital content to the user;
collecting, with a plurality of sensors, response data indicative of a response presented by the user in response to the digital content;
time correlating the response data with the presentation of the digital content; and
producing a response profile that includes the correlated response data with variables presented at the correlated time in the digital content;
providing each of the plurality of response profiles to a machine learning system as training inputs to train the machine learning system to produce a trained machine learning system;
developing, with the trained machine learning system, a plurality of models, each model corresponding to a different user type;
determining a user of a first user type to which an advertisement is to be presented;
providing, to the trained machine learning system, the first user type and candidate digital content components;
determining, with the trained machine learning system, a model of the plurality of models corresponds to the first user type;
producing, based at least in part on the model and the candidate digital content components, an advertisement; and
presenting the advertisement to the user.

15. The method of claim 14, further comprising:
determining at least one condition corresponding to the user; and
wherein the model is further determined based at least in part on the at least one condition.

16. The method of claim 15, wherein the condition includes at least one of a device type of a device, a location of the device, an altitude, a temperature, a weather, an application in which the advertisement is to be presented, a time of day, a day of week, or a month of year.

17. The method of claim 14, further comprising:
collecting, from the user, response data indicative of a response presented by the user in response to the advertisement; and
providing the response data to the trained machine learning system.

18. The method of claim 14, wherein the plurality of users are in a controlled environment in which at least one condition is held constant among each of the plurality of users.

19. The method of claim 14, further comprising:
determining a desired response to the advertisement; and
wherein the model is further determined based on the desired response.

20. The method of claim 14, further comprising:
determining, for the model, a likelihood of engagement with the advertisement by the user of the first user type.
